(19) United States
(12) Patent Application Publication
(10) Pub. No.: US 2019/0365617 A1
(43) Pub. Date: Dec. 5, 2019

(54) RINSE-OFF CLEANSING COMPOSITIONS COMPRISING MATERIALS THAT MODIFY SEBUM

(71) Applicant: The Procter & Gamble Company, Cincinnati, OH (US)

(72) Inventors: Supriya Punyani, Singapore (SG); Ioannis Constantine Constantinides, Wyoming, OH (US); Steven Hardy Page, Lawrenceburg, IN (US); Sandra Nichole Isaacs, Colerain, OH (US)

(21) Appl. No.: 16/209,294

(22) Filed: Dec. 4, 2018

Related U.S. Application Data
(60) Provisional application No. 62/680,822, filed on Jun. 5, 2018.

(51) Int. Cl. A61K 8/34; A61K 8/46; A61K 8/27; A61K 8/25; A61K 8/02; A61K 8/04; A61Q 5/02

(52) U.S. Cl. CPC A61K 8/345; A61K 8/342; A61K 8/463; A61Q 5/02; A61K 8/25; A61K 8/0275; A61K 8/042; A61K 8/27

(57) ABSTRACT

The present invention is directed to a rinse-off cleansing composition comprising from about 0.1 wt. % to about 12 wt. % of a 1,2-diol having a carbon chain with a length of more than 8 carbons; from about 0.1 wt. % to about 10 wt. % of a solid particle, wherein (1) the interfacial tension between the solid particle and sebum is from about 5 to about 18 dyn/cm; (2) the sebum exhibits spreading coefficient on the solid, which is greater than about 22 dyn/cm; and (3) the work of adhesion of the sebum to the solid particle, which is greater than about 75 dyn/cm. and an aqueous carrier.

### FIELD OF THE INVENTION

The present invention relates to rinse-off cleansing compositions comprising 1,2-diols, hydrophobic particles and detersive surfactant that provide effective sebum removal, and durable clean feel on hair and scalp.

### BACKGROUND OF THE INVENTION

Clean Scalp and Hair is described by consumers as having no sticky or greasy feel, no clumped fibers, no odor, and no hair weigh-down. Generally, consumers perceive unclean scalp and hair when sebum in liquid state builds up on their scalp and their hair during the end of the day. The liquid sebum on hair and scalp is often associated with unclean, greasy, oily and dirty look, feel and smell. Sebum is continuously secreted out of the sebaceous glands on scalp in liquid form. Due to dynamic environment (exposure to UV and microflora), sebum is unstable and its composition rapidly changes. As a result, it typically exists in more than one phase on the scalp and it gets transferred to hair. Sebum secretion is a continuous process, sebum re-appears on the scalp with significant amounts accumulating within 5-6 hours after shampooing. As mentioned above, sebum gets progressively transferred to the hair fibers, which leads to unclean consumer perception within 5-6 hours from the previous wash. Thus, a need exists for cleansing products that can effectively remove sebum and delay the appearance and feel of unclean scalp and hair. The inventors of the present invention surprisingly found that hair care products comprising certain 1,2-diols, solid particles along with detersive surfactant can achieve this objective.

Without wishing to be bounded by theory, the presence of the combination of solid particles with certain 1,2-diols in hair care compositions provide for the benefits by modifying the sebum physical properties such as melting characteristic and/or absorbing sebum. As a result, the transfer of sebum from the scalp to hair fibers is reduced, making hair appearance and feel to be less unclean. In addition, treatment the hair and scalp with the inventive rinse-off cleansing compositions contributes to an effective sebum removal.

### SUMMARY OF THE INVENTION

The present invention is directed to a rinse-off cleansing composition comprising:
a) From about 0.1 wt. % to about 12 wt. % of a 1,2-diol having a carbon chain with a length of more than 8 carbons;
b) From about 0.1 wt. % to about 10 wt. % of a solid particle, wherein
   (1) the interfacial tension between the solid particle and sebum is from about 5 to about 18 dyn/cm;
   (2) the sebum exhibits spreading coefficient on the solid, which is greater than about 22 dyn/cm; and
   (3) the work of adhesion of the sebum to the solid particle, which is greater than about 75 dyn/cm;
c) From about 5% to about 35% of one or more detersive surfactant; and
d) an aqueous carrier.

The product compositions of the present invention provide durable clean feel on hair and scalp and easy sebum removal.

### BRIEF DESCRIPTION OF THE DRAWINGS

It is to be understood that both the foregoing general description and the following detailed description describe various non-limiting examples and are intended to provide an overview or framework for understanding the nature and character of the claimed subject matter. The accompanying drawings are included to provide a further understanding of various non-limiting examples, and are incorporated into and constitute a part of this specification. The drawings illustrate various non-limiting examples described herein, and together with the description serve to explain the principles and operations of the claimed subject matter.

FIG. 1 is a non-limiting example depicting the measuring of angle of contact.

### DETAILED DESCRIPTION OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Rinse-off” in reference to compositions, means compositions intended to be applied to hair and/or scalp, spread and massaged on these substrates, followed by rinsing with water.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography. “QS” means sufficient quantity for 100%.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Solid Particles”, as used herein, means materials that are solid at temperature below 30° C. “Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

Shampoos and body washes are considered rinse-off cleansing composition.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

The composition of the present invention provides longevity of hair clean feel and appearance. They also provide excellent cleaning performance. An important element in the rinse-off cleansing composition is the combination of sebum modifiers. This combination can modify the sebum physical properties such as melting characteristic and/or absorbing sebum so that sebum can be prevented from transferring from the scalp to hair fibers over time. The excellent cleansing performance of the inventing composition is determined by measuring the percent removal of sebum from scalp and hair as a result of its use as compared to the corresponding sebum removal achieved by a similar composition which does not comprise the combination of sebum modifiers.

### Sebum Modifier

The combination of the following classes of sebum modifiers can modify the sebum physical properties such as melting characteristic and/or absorbing sebum.
a) 1,2 diols contain having a carbon chain with a length of more than 8 carbons; e.g. 1,2-decanediol, 1,2-dodecanediol, 1,2-octanediol for 1-2-diols.
b) hydrophobic solid particle or hydrophobically modified solid particle such as silica silylate, zinc carbonate, hydrophobic clay, zinc oxide, polyethylene powders, polypropylene powders, polystyrene powders, calcium silicate, polyethylene, nylon, boron nitride, mica, clays such as bentonite, montmorillonite and kaolin, zeolite, cyclodextrins, fumed silica, synthetic clays such as polymer powders including natural, synthetic, and semisynthetic cellulose, fluorocarbon resins, polypropylene, modified starches of cellulose acetate, particulate cross-linked hydrophobic acrylate or methacrylate copolymers and mixtures thereof. Starches hydrophobically modified to have a high capacity for loading oils. Such starches can be modified with alkyl or alkenyl substituted dicarboxylic acids. Such materials may contain counter-ions, for example metals such as aluminum. A preferred such material is Natrasorb HFB available from National Starch and Chemical Company, U.S.A, which contains aluminum starch octenyl succinate. Other suitable materials from National Starch and Chemical include Natrasorb Bath, Dry-Flow PC, Dry-How XT, and Dry-Flow Pure. The surface tension modifying agents are modified protein derivatives that reduce the surface tension of oil. Nonlimiting examples include Vegepol (sodium C8-16 isoalkylsuccinyl soy protein succinate) from Brooks Industries, NJ, and the like.
c) saturated fatty acid contains less than 20 total carbon atoms e.g. stearic acid

The concentration of the 1,2-diol in the rinse-off cleansing composition of the present invention is from about 0.1% to about 12% by weight of the rinse-off cleansing composition. In a rinse-off cleansing composition example, the 1,2-diol is present from about 0.2 to about 5% by weight of the rinse-off cleansing composition; in a further example from about 0.5 to about 4% by weight of the rinse-off cleansing composition; and yet in a further example from about 1.0 to about 3.0%. The concentration of the solid particle in the hair composition of the present invention is from about 0.1% to about 10%. The solid particle is present from about 0.5 to about 5%, more preferably from about 1.0 to about 2.0%.

In an attempt, to identify the materials as sebum modifiers, which can change the physical characteristics of sebum such as their melting temperature, various mixtures of materials with sebum are prepared and measured using differential scanning calorimetry (DSC) method. The following table provides the corresponding measurements.

TABLE 1
Example of Sebum Modifiers

| | Endothermic | Endothermic | Endothermic | Endothermic |
|---|---|---|---|---|
| Sebum Modifier | Temperature | Temperature | Temperature | Temperature |
| Material | Peak 1 (° C.) | Peak 2(° C.) | Peak 3 (° C.) | Peak 4 (° C.) |
| Petrolatum | −23.00 | 3.00 | 16 | 40 |
| 1,2-Dodecanediol | −18.00 | 4.90 | 21 | 36 |
| Stearyl alcohol | −18.00 | 6.00 | 15 | 36 |
| Cetyl alcohol | −18.00 | 4.00 | 18 | 26 |
| 1,2-Decanediol | −20.00 | | | 42 |
| Stearic acid | −18.20 | 6.50 | | 39 |
| Zinc carbonate | 0.5 | 7 | | 75 |
| Zinc stearate | −4 | 8 | 21 | 97 |
| 1,2-octadecanediol | −4 | 10 | 20 | 51 |

In order to identify the appropriate solid particles, the absorption/adsorption characteristics of sebum on various solid particles is measured using contact angle method. The following table provides the corresponding measurements.

TABLE 2
Example of Solid Particles Physical Properties

| | Nonpolar | Polar | Total Surface | | | |
|---|---|---|---|---|---|---|
| | component | component | Tension | Work of | Spreading | Interfacial |
| | of Surface | of Surface | (Polar + | Adhesion | Coefficient | Tension |
| Material | Tension | Tension | Nonpolar) | (sebum) | (sebum) | (sebum) |
| Talc¹ | 22.53 | 5.91 | 28.44 | 55.01 | 1.07 | 0.40 |
| Silica² | 32.87 | 32.20 | 65.08 | 76.37 | 22.43 | 15.68 |
| Silica | 49.07 | 26.14 | 75.21 | 86.71 | 32.77 | 15.47 |
| Silylate³ | | | | | | |
| (hydrophobic | | | | | | |
| silica) | | | | | | |
| Cellulose | 27.50 | 41.00 | 68.50 | 74.24 | 20.30 | 21.23 |
| untreated⁴ | | | | | | |
| Zinc | 44.83 | 26.38 | 71.21 | 83.78 | 29.84 | 14.40 |
| Carbonate | | | | | | |

¹Supplied by Sigma Aldrich
²Supplied by Dow Corning
³Supplied by Dow Corning
⁴Supplied by Sigma Aldrich

### Product Forms

The rinse-off cleansing composition of the present invention comprise (a) 1,2-diol having a carbon chain with a length of more than 8 carbons, (b) solid particles which are able to absorb/adsorb sebum from scalp and/or hair, (c) detersive surfactant, and (d) an aqueous carrier.

### 1,2-Diols

The rinse-off cleansing composition contains from about 0.1 wt. % to about 12 wt. % 1,2-diols having a carbon chain with length of more than 8 carbons. Non-limited examples include 1,2-dodecanediol, 1,2-decanediol, and 1,2-octadecanediol.

Without being limited by theory, such 1,2-diols contribute to the modification of the melting characteristics of sebum, making it more available to be absorbed/adsorbed by the solid particle which is present in the composition.

### Solid Particles

The rinse-off cleansing composition contain from about 0.1 wt. % to about 10 wt. % solid particles, which are able to absorb/adsorb sebum from scalp and/or hair wherein,
(1) the interfacial tension between the solid particle and sebum is from about 5 to about 18 dyn/cm;
(2) the sebum exhibits spreading coefficient on the solid, which is greater than about 22 dyn/cm; and
(3) the work adhesion of the sebum to the solid particle, which is greater than about 75 dyn/cm.

Non-limited examples of such solid particles include zinc carbonate, hydrophobically-modified silica, hydrophobically modified clay, zinc oxide, polyethylene powders, polypropylene powders, polystyrene powders, calcium silicate, polyethylene, nylon, boron nitride, mica, clays such as bentonite, montmorillonite and kaolin, zeolite, cyclodextrins, fumed silica, synthetic clays such as polymer powders including natural, synthetic, and semisynthetic cellulose, fluorocarbon resins, polypropylene, modified starches of cellulose acetate, particulate cross-linked hydrophobic acrylate or methacrylate copolymers and mixtures thereof. Starches hydrophobically modified to have a high capacity for loading oils. Such starches can be modified with alkyl or alkenyl substituted dicarboxylic acids. Such materials may contain counter-ions, for example metals such as aluminum. A preferred such material is Natrasorb HFB available from National Starch and Chemical Company, U.S.A, which contains aluminum starch octenyl succinate. Other suitable materials from National Starch and Chemical include Natrasorb Bath, Dry-Flow PC, Dry-How XT, and Dry-Flow Pure. The surface tension modifying agents are modified protein derivatives that reduce the surface tension of oil. Nonlimiting examples include Vegepol (sodium C8-16 isoalkylsuccinyl soy protein succinate) from Brooks Industries, NJ, and the like.

The rinse-off cleansing composition delivers consumer desired shampooing of the scalp and hair. In addition, the composition may contain scalp anti-dandruff and other scalp actives to deliver anti-dandruff and other scalp health benefits. It may also comprise other optional ingredients such as silicone or organic conditioning agents, hair health actives, and other ingredients.

Other Components in the Rinse-Off Cleansing Composition

A. Aqueous Carrier

The rinse-off cleansing composition comprises an aqueous carrier. Accordingly, the composition can be in the form of a pourable liquid (under ambient conditions). The aqueous carrier is present at a level of at least 40 wt. %, from about 40 wt. % to about 95 wt. %, or from about 60 wt. % to about 85 wt. %. The aqueous carrier may comprise water, or a combination of water and an water-miscible organic solvent. In one aspect may comprise water with minimal or no significant concentrations of organic solvent, except as otherwise incidentally incorporated into the composition as minor ingredients of other components. Non-limiting examples of water-miscible solvents include lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

B. Detersive Surfactant

The rinse-off cleansing composition comprises one or more detersive surfactants, which provides cleaning performance to the composition. The one or more detersive surfactants in turn may comprise an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety.

The concentration of the detersive surfactant component in the shampoo composition should be sufficient to provide the desired cleaning and lather performance, and generally ranges from about 5 wt. % to about 40 wt. %, from about 8 wt. % to about 35 wt. %, from about 10 wt. % to about 25 wt. %, from about 12 wt. % to about 20 wt. %.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the shampoo composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate, sodium lauroyl methyl isethioniate, sodium cocoyl glutamate, sodium lauroyl glutamate and combinations thereof. The anionic surfactant can be sodium lauryl sulfate or sodium laureth sulfate.

Suitable amphoteric or zwitterionic surfactants for use in the shampoo composition herein include those which are known for use in shampoo or other personal care cleansing. Concentrations of such amphoteric surfactants range from about 0.5 wt. % to about 20 wt. %, and from about 1 wt. % to about 10 wt. %. Non-limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the shampoo composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present shampoo composition include cocoamphoacetate, cocoamphodiacetate, lauroamphoacetate, lauroamphodiacetate, and mixtures thereof.

Zwitterionic detersive surfactants suitable for use in the shampoo composition include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Zwitterionics such as betaines can also be selected.

Non-limiting examples of other anionic, zwitterionic, amphoteric or optional additional surfactants suitable for use in the shampoo composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

C. Gel Network

The rinse-off cleansing composition may also comprise a gel network. The gel network comprises (i) from about 0.1% to about 20% of one or more fatty alcohols, alternative from about 0.5% to about 14%, alternatively from about 1% to about 10%, alternatively from about 6% to about 8%, by weight of the gel network; (ii) from about 0.1% to about 10% of one or more gel network surfactants, by weight of the gel network; and (iii) from about 20% to about 95% of an aqueous carrier, alternatively from about 60% to about 85% by weight of the gel network.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Non-limiting examples of fatty alcohols include, cetyl alcohol, stearyl alcohol, behenyl alcohol, and mixtures thereof. Mixtures of cetyl and stearyl alcohol in a ratio of from about 20:80 to about 80:20 are suitable.

The gel network surfactants may be any of the detersive surfactants described in the detersive surfactant section herein.

The aqueous carrier of the gel network may comprise water, or a combination of water and a water-miscible organic solvent. In one aspect may comprise water with minimal or no significant concentrations of organic solvent, except as otherwise incidentally incorporated into the composition as minor ingredients of other components. Non-limiting examples of water-miscible solvents include lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

Other Components

### Silicone Conditioning Agent

The compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, epoxy groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the polymer can be in an emulsified form with droplet size of about 10 nm to about 30 micrometers Other solid or semi-solid conditioning agents may be present in the composition including high melting temperature fatty alcohols, acids, esters, amides or oligomers from unsaturated esters, alcohols, amides. The oligomeric esters may be the result of oligomerization of naturally-occurring unsaturated glyceride esters. Such solid or semi-solid conditioning agents may be added or present as mixtures with organic oils.

### Nonionic Polymers

The rinse-off cleansing composition of the present invention may also further comprise a nonionic polymer. The conditioning agent for use in the hair care composition of the present invention may include a polyalkylene glycol polymer. For example, polyalkylene glycols having a molecular weight of more than about 1000 are useful herein. Useful are those having the following general formula (VIII) wherein R11 is selected from the group consisting of H, methyl, and mixtures thereof; and v is the number of ethoxy units. The polyalkylene glycols, such as polyethylene glycols, can be included in the hair care compositions of the present invention at a level of from about 0.001 wt. % to about 10 wt. %. The polyethylene glycol may be present in an amount up to about 5 wt. % based on the weight of the composition. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

### Organic Conditioning Materials

The conditioning agent of the compositions of the present invention may also comprise at least one organic conditioning material such as oil or wax, either alone or in combination with other conditioning agents, such as the silicones described above. The organic material can be non-polymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

### Deposition Aids

The rinse-off cleansing compositions of the present invention may further comprise a deposition aid, such as a cationic polymer. Cationic polymers useful herein are those having an average molecular weight of at least about 5,000, alternatively from about 10,000 to about 10 million, and alternatively from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

The cationic polymer can be included in the hair care compositions of the present invention at a level of from about 0.001 wt. % to about 10 wt. %. The cationic polymer may be present in an amount up to about 5 wt. % based on the weight of the composition.

### Benefit Agents

The rinse-off cleansing composition may further comprise one or more additional benefit agents. The benefit agents comprise a material selected from the group consisting of anti-dandruff agents, anti-fungal agents, anti-itch agents, anti-bacterial agents, anti-microbial agents, moisturization agents, anti-oxidants, vitamins, lipid soluble vitamins, chelants, perfumes, brighteners, enzymes, sensates, attractants, dyes, pigments, bleaches, and mixtures thereof.

In one aspect said benefit agent may comprise an anti-dandruff agent. Such anti-dandruff particulate should be physically and chemically compatible with the components of the composition, and should not otherwise unduly impair product stability, aesthetics or performance Non-limiting examples of anti-dandruff agents suitable for use in rinse-off cleansing compositions include pyridinethione salts, azoles (e.g., ketoconazole, econazole, and elubiol), selenium sulfide, particulate sulfur, salicylic acid, and mixtures thereof. A common anti-dandruff agent is pyridinethione salt. Hair care compositions can also include a zinc-containing layered material. An example of a zinc-containing layered material can include zinc carbonate materials. Of these, zinc carbonate and pyridinethione salts (particularly zinc pyridinethione or “ZPT) are common in the composition, and often present together.

### Rheology Modifier

The rinse-off cleansing composition may include one or more rheology modifiers to adjust the rheological characteristics of the composition for better feel, in-use properties and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. The leave-on treatment may comprise from about 0.01% to about 3% of a rheology modifier, alternatively from about 0.1% to about 1% of a rheology modifier,

The one or more rheology modifier may be selected from the group consisting of polyacrylamide thickeners, cationically modified polysaccharides, associative thickeners, and mixtures thereof. Associative thickeners include a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. These materials may have a hydrophobe that can be selected from cetyl, stearyl, oleayl, and combinations thereof, and a hydrophilic portion of repeating ethylene oxide groups with repeat units from 10-300, alternatively from 30-200, and alternatively from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of additional rheology modifiers include acrylamide/ammonium acrylate copolymer (and)polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-2 hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquarternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and/or combinations thereof.

### pH of the Composition

The rinse-off cleansing compositions may also comprise one or more pH adjusting material. The compositions may have a pH in the range from about 2 to about 10, at 25° C. The shampoo composition may have a pH in the range of from about 2 to about 6, alternatively from about 3.5 to about 5, alternatively from about 5.25 to about 7.

The hair care compositions mentioned above may further comprise one or more pH buffering agent. Suitable buffering agents are well known in the art and include for example ammonia/ammonium acetate mixture and monoethanolamine (MEA). The shampoo composition may comprise citric acid, wherein the citric acid acts as a buffer.

### Evaluation Methods

A. Sample Preparation for Differential Scanning Calorimetry (DSC) Measurement

A quantity of 1 g of artificial sebum is mixed with 1 g of 1,2-diol sebum modifier material at room temperature and heated at 40° C. water bath for 2 minutes to a solution or a uniform mixture and the solution or uniform mixture is cooled at room temperature. The sample thus prepared is measured using differential scanning calorimetry method as described below.

B. Differential Scanning Calorimetry (DSC) Measurement

A DSC 204 Netzsch TASC 414/3A is used for the evaluation of 1,2-diol sebum modifiers. Each measurement is performed in triplicate. The samples consists of approximately 5.5 mg of 1:1 sebum: sebum modifier, as prepared by the method described in A above. The sample is placed into T-Zero aluminum DSC pans, and then covered with stainless steel meshes. A sand baseline and burn off is performed before running samples, and after every 10 samples. Each evaluation is conducted by varying the temperature of the sample between the values of −50° C. to 300° C. at a heating rate of 5° C./minute under 200 ml/min nitrogen purge. An empty pan of the same type is employed as a reference and tested under the same experimental conditions. The standard deviation of this method is less than 5%. Temperature measurements are taken at the endotherm peaks of physical changes in sebum components and curve-fitting energy integration is performed for the entire endotherm curve.

C. Measurement of Solid Particles Physical Properties Using Contact Angle Method

A series of physical parameters of solid surfaces are important for selecting the materials appropriate materials for sebum modification.
a. Dispersion component of the surface tension. This is the component of solid surface tension that is related to intermolecular attraction caused by nonpolar dispersion forces.
b. Polar component of the surface tension. This is the component of solid surface tension that is related to polar forces, such as hydrogen bonding and ion-dipole forces.
c. Solid surface tension. This is the surface tension of a solid surface. This parameter cannot be measured directly, but must be determined by extrapolation of polymer melt or solution data to 100% solids or by contact angles with liquids of known surface tension.
d. Surface energy. This is the excess free energy of surface molecules compared to those of the bulk material. It arises from unbalanced molecular cohesive forces at a surface that cause the surface to contract and behave like a film or membrane. The surface energy is expressed in energy/unit area, such as joules/cm².
e. Surface tension. This expresses the force necessary to break the surface of a film of a given length (units are force/length, such as dyn/cm or newtons/m); the same numerically as surface energy, but different units.

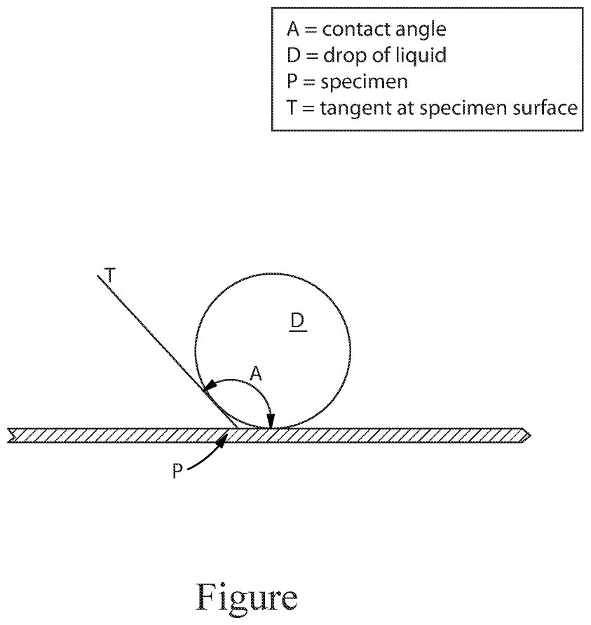

The measurement of the contact angle on solid particles can be used to determine the above-mentioned physical properties. Contact angle is the interior angle that a drop makes between the substrate and a tangent drawn at the intersection between the drop and the substrate as shown in FIG. 1. This is the angle formed by a liquid at the three-phase boundary where a liquid, gas (air) and solid intersect.

Contact angles are determined using a ASTM D7490-13 (Standard Test Method for Measurement of the Surface Tension of Solid Coatings, Substrates and Pigments using Contact Angle Measurements).

The equipment used includes a goniometer consisting of a controlled light source, a stage to hold the tile, and a microscope or camera for viewing of the drop on the tile is required (First Ten Angstrom, Model 200, or equivalent). A 1-mL hypodermic syringe is also used equipped with a No. 27 blunt tipped stainless-steel needle, capable of providing 100 to 200 drops from 1 mL. For this evaluation, the following reagents are used: (a) water—Type II reagent water (distilled) in accordance with ASTM Specification D1193-99, and (b) diiodomethane (99+% purity).

The method involves the following steps:
1. Formation of a pellet of solid particles on a ceramic tile. The ceramic tile should not be touched with the fingers or contaminated in any other way during position on goniometer stage.
2. Measurement of the contact angle on the solid particles in a constant temperature (73±2° F.) and humidity environment (50±5%) using the goniometer. Contact angles are measured for each discrete droplet of water and diiodomethane on the tile as described in ASTM D7334 (or the manufacturer's literature for the specific instrument used). More specifically,
   (a) The tile is positioned so that a drop of the liquid can be deposited without visible distortion of the drop shape due to movement.
   (b) The tip of the hypodermic needle is set at the distance from the surface recommended by the manufacturer of the instrument (3 mm (⅛ inches for the specific instrument) and a drop of the test liquid 5 μL in size is deposited on the tile. The drop size should be controlled to ±0.1 μL.
   (c) The camera or video device is focused so that the image of the drop can be captured.
   (d) Two contact angle measurements (one on each drop edge) are made for a water droplet on the tile using commercial software designed to extract contact angles from movies or images. For example, First Ten Angstrom software version 2.1, build 363, or equivalent. If the contact angles on two edges are different by more than 4°, the values are eliminated and the test is repeated. The measurement is repeated 5 more times on new droplets. The contact angle for the tile is the average of the six angles measured for each side.

The image acquisition speed should capture at least 10 images from the time the drop hits the surface to the time it cannot be resolved from the surface of the sample. For the measurements reported herein, a capture rate of 900 images per second is utilized. The software described above extracts the contact angles from the video feed. The volume is also calculated using the same software under the sessile volume. The contact angles are plotted with the sessile volume plots. Enough time is allowed for the drop to wet out to equilibrium. However, in highly absorptive systems the drop absorbs into the material before equilibrium is achieved. In these cases, in which the drop rapidly (<0.2 seconds) absorbs into the substrate, video is progressed until 2% of the volume of the drop absorbed into the substrate. The contact angle is recorded at that time point. This might mean the first resolved image in extremely fast absorbing systems if the second image shows more than 2% volume loss.
(e) The measurement of step (d) is repeated for diiodomethane droplet (instead of water droplet). The measurement is performed on the finished side of the clean untreated ceramic tile.
3. Calculation of physical properties of solids

The contact angle values for water and diiodomethane are substituted into two separate expressions of the Owens-Wendt-Kaelble equation (one for each liquid). This results in three equations and two unknowns, which are then solved for the dispersion and polar components of surface tension (refer below paragraph for the equations and calculations).

### Calculation of Surface Energy

The Owens-Wendt-Kaelble equation:

$${σ}_{lg}^{T} \frac{( cos θ + 1 )}{2} = {( {σ}_{lg}^{D} {γ}_{sg}^{D} )}^{1 / 2} + {( {σ}_{lg}^{P} {γ}_{sg}^{P} )}^{1 / 2}$$

where:
θ=the average contact angle for the test liquid on the test specimen,
σ_{lg}^{T}=the total surface tension of the test liquid in dyn/cm
σ^{D} and σ^{P}=the dispersive and polar components of the liquid surface tension, respectively, also in dyn/cm.
σ_{sg}=the total surface energy of the test substrate in dyn/cm
σ^{D} and σ^{P}=the dispersive and polar components of the test substrate, respectively, also in dyn/cm.

| | Surface Tension (σ_{lg}) (dyn/cm) | | |
|---|---|---|---|
| Solvent | Nonpolar | Polar | Total |
| Diiodomethane | 50.8 | 0 | 50.8 |
| Water | 21.8 | 51.0 | 72.8 |

The Owens-Wendt-Kaelble equation is simplified to the following equation when a dispersive (nonpolar) solvent such as diiodomethane is used:

$${σ}_{lg}^{T} \frac{( cos θ + 1 )}{2} = {( {σ}_{lg}^{D} {γ}_{g}^{D} )}^{1 / 2}$$

The dispersive (nonpolar) component of surface energy (σ^{D}_{sg}) is determined. Surface tension properties for diiodomethane are known and included in the table above. The contact angle is experimentally determined using the method delineated above.

Upon inserting the calculated dispersive component of surface energy (σ^{D}_{sg}) for the substrate into the Owens-Wendt-Kaelble equation delineated above and using the contact angles determined for water, the polar component of surface energy (σ^{P}_{sg}) of the substrate is determined because the surface tension properties for water are known and included in the table above. The dispersive component (σ^{D}_{sg}) of the substrate is determined with diiodomethane as explained above.

### Calculation of Thermodynamic Parameters

Thermodynamic parameters is calculated by inserting surface energy components into the following equations of state:

### Spreading Coefficient:

The spreading coefficient (S) is determined by de Gennes (de Gennes, P.-G., Reviews of Modern Physics (1985), 57, 827-863).

S=γ_{sg}^{T}−σ_{lg}^{T}−σₛₗ

Where σₛᵢ is the interfacial tension

### Interfacial Tension:

The Owens-Wendt equation of state is used to determine interfacial tension σₛᵢ (D. K. Owens and R. C. Wendt, Journal of Applied Polymer Science (1969), 13, 1741-1747).

σₛₗ^{T}=γ_{sg}^{T}+σ_{lg}^{T}−2(σ_{lg}^{D}γ_{sg}^{D})^{1/2}−2(σ_{lg}^{P}γ_{sg}^{P})^{1/2}

### Work of Adhesion:

The work of adhesion (W) using the Dupré equation of state (A. Dupré, Theorie Mechanique de la Chaleur; Gauthier-Villars: Paris, 1869; pp 36W).

W=γ_{sg}^{T}+σ_{lg}^{T}−σₛₗ

D. Method of Determination of Sebum Removal

Method of Preparing Sebum-Fluorescence Dye

An amount of 20 g of artificial sebum is mixed with 0.03 g of Tinopal B (Benzoxazole, 2,2′-(2,5-thiophenediyl) bis[5-(1,1-dimethylethyl)], from BASF). The mixture is prepared in a brown vial to prevent light exposure and heated to 54° C. using water bath to melt and mix the components. Artificial Sebum composition is prepared by adding all the materials mentioned in below table and then heated to 54° C. using a water bath, to make it a uniform mixture.

| | | Amount added |
|---|---|---|
| Raw Material | Weight % | (for 100 g) |
| Stearic Acid | 14 | 14 |
| Oleic acid | 8 | 8 |
| Squalene | 12 | 12 |
| Cetyl Palmitate | 12 | 12 |
| Isostearyl Isostearate | 12 | 12 |
| Trioctanoin | 20 | 20 |
| Caprylic Capric | 20 | 20 |
| Triglyceride | | |
| Cholesterol | 2 | 2 |

### Scalp Mimic (Bioskin) Used for Sebum Removal Method

Bioskin called as scalp mimic is disc shape (Diameter 50 mm×thickness 5 mm). It is made up of urethane-elastomer has a natural skin-like touch. Bioskin is available from Bioskin Net (http://www.bioskin.ne.jp/english/makeup/foundation.html).

Method of Treating Scalp Mimic with Shampoo Composition

A scalp mimic is weighed using an analytical balance at temperature of 25° C. and at 50% relative humidity and its image is acquired by a camera while the scalp mimic is illuminated by UV light with wavelength of 256 nm and power of 8 W. An amount of 0.10 g of the Sebum-dye uniform mixture is then applied and spread onto the scalp mimic disc. This amount corresponds to a dosage of 0.1 g of sebum-dye mixture per gram of the scalp mimic. The scalp mimic is weighed again and its image is acquired again under the same conditions as mentioned above. The weighing and the image acquisition is performed immediately after the application of the sebum-dye mixture (image at to). The sebum-dye mixture imparts blue color under the UV light, which enables the determination of the quantity of the sebum on the scalp mimic. Then, the scalp mimic is wetted with 0.1 g of water and a quantity of 0.1 g of shampoo is applied. The shampoo is massaged onto the scalp mimic for 30 seconds and rinsed with deionized water for 10 seconds at a flow rate of 20 ml/min. The scalp mimic is then allowed to air dry, weighed again and its image is acquired under the conditions described above (image at t_{w}). The images of the scalp mimic are acquired using a digital single-lens reflex Camera with parallel polarizers.

The area of the image occupied by blue color intensity light due to sebum-dye mixture is analyzed (selecting the entire scalp mimic) using 2D projection. The analysis is performed using a Java-based image processing program. Then, the mean projected area is determined for the scalp mimic at to time (corresponding to area Aₜ₀) and for the scalp mimic at t_{w} time (corresponding to area A_{tw}). Then, the sebum removal is calculated using the equation given below. Each experiment is repeated with three scalp mimic discs.

% Sebum Removal=100×(A_{tw}/Aₜ₀).

The standard error of sebum removal method is less than 10%.

### Shampoo Hair Treatment Protocol:

All testing for clean feel and clean appearance is performed on Virgin Caucasian Hair weighing approximately 2.0 grams and having a length of approximately 6 inches. The hair switches are commercially available from IHIP (International Hair Importers). Each evaluation is performed in three hair switches and the reported results represent averages. An amount of 14.4 μL of sebum is spread onto separate hair switch using cotton pad. That is, the dosage is 7.2 μL of artificial sebum per g of hair. Then the rinse-off shampoo is applied according to the following protocol. An amount of 0.40 g of shampoo is spread via a syringe onto separate hair switch. That is, the dosage is 0.20 g of shampoo per gram of hair. Each application consists of adding shampoo to the hair, milking for 30 seconds followed by rinsing for 30 seconds. Excess water is squeezed from the hair switches. This protocol is repeated only once. Hair is air-dried overnight at room temperature, 50% RH. Aritificial sebum is reapplied, 14.4 μL of sebum is spread onto hair switch using cotton pad.

E. Evaluation of Hair Switch Clean Feel and Clean Appearance

The treated hair switches are air dried and rated by ten expert graders in terms of (a) clean (non-greasy) appearance and (2) feel based on a 5-point scale, 0 being the best clean (no greasy) and 5 being the worst clean (very greasy) separately.

### EXAMPLES AND COMPOSITIONS

The following examples illustrate the invention described herein. The exemplified shampoo compositions can be prepared by conventional formulation and mixing techniques. It will be appreciated that other modifications of the shampoo compositions within the skill of those in the formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

### Shampoo Compositions

| | Comparative | | | | | | |
|---|---|---|---|---|---|---|---|
| | Ex. A | EX 1 | Ex. 2 | Ex. 3 | Ex. 4 | Ex. 5 | Ex. 6 |
| Ingredients | (wt./wt.) % | (wt./wt.) % | (wt./wt.) % | (wt./wt.) % | (wt./wt.) % | (wt./wt.) % | (wt./wt.) % |
| Water Purified | Q.S to | Q.S to | Q.S to | Q.S to | Q.S to | Q.S to | Q.S to |
| | 100 | 100 | 100 | 100 | 100 | 100 | 100 |
| Sodium Laureth 3 | 21.6 | 21.6 | 21.6 | 21.6 | 21.6 | 21.6 | 21.6 |
| Sulfate 28% solution | | | | | | | |
| Sodium Lauryl Sulfate | 34.5 | 34.5 | 34.5 | 34.5 | 34.5 | 34.5 | 34.5 |
| 29% solution | | | | | | | |
| Laureth-4 | 0.9 | 0.9 | 0.9 | 0.9 | 0.9 | 0.9 | 0.9 |
| Dimethicone 330M cps | 0.5 | 0.5 | 0.5 | 0.5 | 0.5 | 0.5 | 0.5 |
| Glycol Distearate | 1.5 | 1.5 | 1.5 | 1.5 | 1.5 | 1.5 | 1.5 |
| Polyquaternium-6 | 0.32 | 0.32 | 0.32 | 0.32 | 0.32 | 0.32 | 0.32 |
| Trisodium | 0 | 0 | 0 | 0 | 0 | 0 | 0 |
| ethylenediamine | | | | | | | |
| disuccinate | | | | | | | |
| Sodium Benzoate | 0.27 | 0.27 | 0.27 | 0.27 | 0.27 | 0.27 | 0.27 |
| Citric acid 50% | 0.52 | 0.52 | 0.52 | 0.52 | 0.52 | 0.52 | 0.52 |
| Solution | | | | | | | |
| Methylchloroisothiazolinone/ | 0.035 | 0.035 | 0.035 | 0.035 | 0.035 | 0.035 | 0.035 |
| methylisothiazolinone | | | | | | | |
| Sodium chloride | 1.66 | 1.66 | 1.66 | 1.66 | 1.66 | 1.66 | 1.66 |
| Fragrance | 0.65 | 0.65 | 0.65 | 0.65 | 0.65 | 0.65 | 0.65 |
| DL-Panthenol 56% | 0.05 | 0.05 | 0.05 | 0.05 | 0.05 | 0.05 | 0.05 |
| solution | | | | | | | |
| Panthenyl Ethyl ether | 0.03 | 0.03 | 0.03 | 0.03 | 0.03 | 0.03 | 0.03 |
| Zinc Carbonate | 0 | 1.0 | 0.0 | 1.0 | 0.0 | 0.0 | 0 |
| 1,2-decanediol¹ | 0 | 0 | 1.0 | 1.0 | 0 | 0 | 0.5 |
| Bentonite² | 0 | 0 | 0 | 0 | 1.0 | 0 | 0 |
| Silica Silylate³ | 0 | 0 | 0 | 0 | 0 | 0.5 | 0.25 |
| % Sebum Removal at | 77 | 82 | 87 | 95 | 81 | 82 | 90 |
| dose of 0.10 g of | | | | | | | |
| composition for each | | | | | | | |
| scalp mimic | | | | | | | |
| Hair feels clean (0 | 4 | 3 | 2 | 1 | 3 | 2 | 2 |
| being Clean (non-greasy) | | | | | | | |
| and 5 being Not Clean | | | | | | | |
| (very greasy) | | | | | | | |
| Hair looks clean (0 | 4 | 3 | 2 | 1 | 3 | 2 | 2 |
| being Clean (non-greasy) | | | | | | | |
| and 5 being Not Clean | | | | | | | |
| (very greasy) | | | | | | | |

¹Symclariol Supplied by. Symrise
²Supplied by Parchem Chemicals, New York
³Supplied by Dow Corning

### Composition Examples 1 to 6 Show Increase in % Sebum Removal Compared to Control Shampoo Composition (Comparative Ex. A)

The feel assessment results indicate that combinations of
(a) 1,2-decanediol and zinc carbonate;
(b) 1,2-decanediol and hydrophobic silica;
   provide, not only sebum removal (resulting in clean benefit), but also clean feel benefit. This is shown by the feel comparison of (a) Example 3 versus Example 1 (b) Example 6 versus Example 4.

### Examples of Shampoos Containing Gel Network

The gel network is prepared separately before it is combined with the other components of the shampoo.

### Shampoo Compositions Containing Gel Network

A vessel is charged with water and the water is heated to about 74° C. Cetyl alcohol, stearyl alcohol, and SLES surfactant are added to the heated water. After incorporation, the resulting mixture is passed through a heat exchanger where the mixture is cooled to about 35° C. Upon cooling, the fatty alcohols and surfactant crystallized to form a crystalline gel network. Example A provides the components and their respective amounts for the gel network composition.

### Gel Network Example 7

| Gel network components | Wt % |
|---|---|
| Water | 76.9 |
| Cetyl alcohol | 4.3 |
| Stearyl alcohol | 7.7 |
| Sodium laureth-3 sulfate (28% Active) | 11 |

| | Ex. 8 | Ex. 9 | Ex. 10 |
|---|---|---|---|
| | (wt./ | (wt./ | (wt./ |
| Ingredients | wt.) % | wt.) % | wt.) % |
| Water Purified | Q.S to 100 | Q.S to 100 | Q.S to 100 |
| Sodium laureth-1 sulfate | 14.9 | 14.9 | 14.9 |
| Guar hydroxypropyltrimonium | 0.33 | 0.33 | 0.33 |
| chloride | | | |
| Polyquaternium-10 | 0.078 | 0.078 | 0.078 |
| Sodium chloride | 0.69 | 0.69 | 0.69 |
| 1,2-decanediol | 2.0 | 0 | 2.0 |
| Zinc carbonate | 0 | 1.0 | 1.0 |
| Tetrasodium EDTA tetrathydrate | 0.16 | 0.16 | 0.16 |
| Sodium benzoate | 0.25 | 0.25 | 0.25 |
| Glycerin | 0.50 | 0.50 | 0.50 |
| Dimethiconol and Dimethicone | 1.00 | 1.00 | 1.00 |
| Sodium xylenesulfonate | 0.42 | 0.42 | 0.87 |
| Polyquaternium-6 | 0.08 | 0.08 | 0.08 |
| Trihydroxystearin | 0.06 | 0.06 | 0.06 |
| Gel network from Example 7 | 18 | 18 | 18 |
| Cocamidopropyl Betaine | 1.70 | 1.70 | 1.70 |
| Perfume | 0.90 | 0.90 | 0.90 |

### Additional Examples/Combinations

A. A rinse-off cleansing composition comprising:
   a) from about 0.1 wt. % to about 12 wt. % of a 1,2-diol having a carbon chain with a length of more than 8 carbons;
   b) from about 0.1 wt. % to about 10 wt. % of solid particles, wherein
      (1) the interfacial tension between the solid particle and sebum is from about 5 to about 18 dyn/cm;
      (2) the sebum exhibits spreading coefficient on the solid, which is greater than about 22 dyn/cm; and
      (3) the work of adhesion of the sebum to the solid particle, which is greater than about 75 dyn/cm.
   c) from about 5 wt % to about 40 wt % of one or more detersive surfactant; and
   d) from about 40% to about 95% of an aqueous carrier.
B. A rinse-off cleansing composition according to Paragraph A, wherein the solid particles is selected from the solid particle is selected from zinc carbonate, hydrophobically-modified silica, hydrophobically modified clay, zinc oxide, polyethylene powders, polypropylene powders, polystyrene powders, calcium silicate, nylon, boron nitride, mica, zeolite, cyclodextrins, fumed silica, synthetic clays, fluorocarbon resins, polypropylene modified starches of cellulose acetate, particulate cross-linked hydrophobic acrylate or methacrylate copolymers and mixtures thereof.
C. A rinse-off cleansing composition according to Paragraph A-B, wherein the solid particle is selected from the group consisting of talc, silica, silica silylate (hydrophobic silica), cellulose untreated, zinc carbonate and mixtures thereof.
D. A rinse-off cleansing composition according to Paragraph A-C, wherein the composition comprises from about 0.2 wt % to about 5 wt. % of the 1,2-diol having a carbon chain length of more than 8 carbons.
E. A rinse-off cleansing composition according to Paragraph A-D, wherein the composition comprises from about 0.5 wt. % to about 4 wt. % of the 1,2-diol having a carbon chain length of more than 8 carbons.
F. A rinse-off cleansing composition according to Paragraph A-E, wherein the composition comprises from about 1 wt. % to about 3 wt. % of the 1,2-diol having a carbon chain length of more than 8 carbons.
G. A rinse-off cleansing composition according to Paragraph A-F, wherein the composition comprises from about 0.5 wt. % to about 5 wt. % of the solid particle.
H. A rinse-off cleansing composition according to Paragraph A-G, wherein the composition comprises from about 1 wt. % to about 2 wt. % of the solid particle.
I. A rinse-off cleansing composition according to Paragraph A-H, wherein the composition comprises from about 40 wt. % to about 95 wt. % of the aqueous carrier.
J. A rinse-off cleansing composition according to Paragraph A-I, wherein the composition comprises from about 60 wt. % to about 85 wt. % of the aqueous carrier.
K. A rinse-off cleansing composition according to Paragraph A-J, wherein the composition further comprises from about 5% to about 50% by the weight of the rinse-off cleansing composition of a gel network composition, wherein the gel network comprises
   a. from about 0.1% to about 20% fatty alcohol by weight of the gel network composition; and
   b. from about 0.1% to about 10% fatty alcohol by weight of the gel network composition; and
   c. from about 20% to about 95% of an aqueous carrier.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular examples of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A rinse-off cleansing composition comprising:
a) from about 0.1 wt. % to about 12 wt. % of a 1,2-diol having a carbon chain with a length of more than 8 carbons;
b) from about 0.1 wt. % to about 10 wt. % of solid particles, wherein
(1) the interfacial tension between the solid particle and sebum is from about 5 to about 18 dyn/cm;
(2) the sebum exhibits spreading coefficient on the solid, which is greater than about 22 dyn/cm; and
(3) the work of adhesion of the sebum to the solid particle, which is greater than about 75 dyn/cm.
c) from about 5 wt. % to about 40 wt. % of one or more detersive surfactant; and
d) from about 40% to about 95% of an aqueous carrier.

2. A rinse-off cleansing composition according to claim 1 wherein the solid particles is selected from the solid particle is selected from zinc carbonate, hydrophobically-modified silica, hydrophobically modified clay, zinc oxide, polyethylene powders, polypropylene powders, polystyrene powders, calcium silicate, nylon, boron nitride, mica, zeolite, cyclodextrins, fumed silica, synthetic clays, fluorocarbon resins, polypropylene modified starches of cellulose acetate, particulate cross-linked hydrophobic acrylate or methacrylate copolymers and mixtures thereof.

3. A rinse-off cleansing composition according to claim 2 wherein the solid particle is selected from the group consisting of talc, silica, silica silylate (hydrophobic silica), cellulose untreated, zinc carbonate and mixtures thereof.

4. A rinse-off cleansing composition according to claim 1 wherein the composition comprises from about 0.2 wt. % to about 5 wt. % of the 1,2-diol having a carbon chain length of more than 8 carbons.

5. A rinse-off cleansing composition according to claim 1 wherein the composition comprises from about 0.5 wt. % to about 4 wt. % of the 1,2-diol having a carbon chain length of more than 8 carbons.

6. A rinse-off cleansing composition according to claim 1 wherein the composition comprises from about 1 wt. % to about 3 wt. % of the 1,2-diol having a carbon chain length of more than 8 carbons.

7. A rinse-off cleansing composition according to claim 1 wherein the composition comprises from about 0.5 wt. % to about 5 wt. % of the solid particle.

8. A rinse-off cleansing composition according to claim 1 wherein the composition comprises from about 1 wt. % to about 2 wt. % of the solid particle.

9. A rinse-off cleansing composition according to claim 1 wherein the composition comprises from about 40 wt. % to about 95 wt. % of the aqueous carrier.

10. A rinse-off cleansing composition according to claim 1 wherein the composition comprises from about 60 wt. % to about 85 wt. % of the aqueous carrier.

11. A rinse-off cleansing composition according to claim 1 wherein the composition further comprises from about 5% to about 50% by the weight of the rinse-off cleansing composition of a gel network composition, wherein the gel network comprises
a. from about 0.1% to about 20% fatty alcohol by weight of the gel network composition; and
b. from about 0.1% to about 10% fatty alcohol by weight of the gel network composition; and
c. from about 20% to about 95% of an aqueous carrier.
